(19) United States
(12) Patent Application Publication
(10) Pub. No.: US 2009/0280171 A1
(43) Pub. Date: Nov. 12, 2009

(54) METHOD OF TREATMENT WITH PREDICTABLY BREAKABLE PHARMACEUTICAL TABLETS

(75) Inventor: Lawrence Solomon, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(73) Assignee: ACCU-BREAK Technologies, Inc., Plantation, FL (US)

(21) Appl. No.: 12/297,017

(22) PCT Filed: Apr. 13, 2007
(86) PCT No.: PCT/US07/66637

§ 371 Date: Oct. 13, 2008

Related U.S. Application Data
(60) Provisional application No. 60/791,834, filed on Apr. 13, 2006.

(51) Int. Cl. A61K 9/20; A61K 31/56

(52) U.S. Cl. 424/464; 514/169

(57) ABSTRACT

The invention involves the use of finished dosage forms, e.g., tablets, by breaking or otherwise dividing them to produce a predictably accurate smaller or lower dose.

### CROSS-REFERENCE TO RELATED APPLICATIONS

This is a Section 371 national filing in the United States Patent and Trademark Office claiming priority to International Application No. PCT/US2007/066637, filed Apr. 13, 2007, which claims the benefit of the filing date of U.S. Provisional Application Ser. No. 60/791,834, filed Apr. 13, 2006.

### BACKGROUND

It is known to create scored tablets with patterns including bisection, trisection and quadrisection. A score is a debossed line or mark, or set of marks, formed into one or more faces of a tablet. While scores may be popularly utilized as a guide for breaking a scored tablet to create a dose which is smaller than the whole dosage form from which it derives, authoritative guides that instruct, or even explicitly permit, a user to utilize the score as a guide for breaking scored tablets are lacking.

An example is the Product Information (“PI” or “label”) for a brand of the anti-convulsant lamotrigine, marketed as Lamictal® in the United States as scored 25 and 100 mg tablets. Despite extremely detailed titration recommendations to physicians in the product label, those recommendations do not include any instruction for the patients, or anyone else such as pharmacists, to split the tablets. The accompanying Lamictal PI, instruction #6, states: “Tablets should be swallowed whole.” This and numerous other examples of manufacturers of scored tablets not recommending breaking through the score in those tablets to provide lower doses useful in dose titrations demonstrate that such action is an “off-label” use.

Given that, inter alia, (1) drug product information (e.g., labeling) has not before indicated that broken portions of scored tablets could be used in place of separately-produced dosage forms of the strength implied by the scoring pattern, (2) in view of the current European Pharmacopoeia's (“EP”) requirement that scored tablets must accurately break into a predictable strength of a dosage forms that meets applicable regulatory standards for separately made dosage forms; and (3) in view of the pending withdrawal of this EP requirement due to the difficulty the pharmaceutical industry has had in meeting its strictures, it is clear that there can be no assurance of what that lower dose is in a portion of a scored tablet that has been broken through the score to provide a lower dose in that portion, such portion of a broken tablet being referred to herein as a tablette, See, for example, Solomon and Kaplan, published International patent applications WO 2005/112898 and WO 2005/112900. It has until now therefore been unachievable to propound a method of dose escalation (or de-escalation) that involves utilizing a half, a third, or a quarter of a dosage form such as a tablet.

It is important to note that patients and other persons have broken scored and unscored tablets to provide lower doses. However, these breaking activities generally could not predictably or reliably create an accurate lower dose. Previous methods of breaking tablets could not assure a patient was reliably ingesting a 20 mg dose from a halved 40 mg tablet. Rather, previous compositions and methods could only provide two doses that are each less than the dose of the whole tablet (taking note of the loss of mass due to crumbling or chipping on tablet breaking). In the case of the subject invention, e.g., breaking of a tablet into two or more tablettes, such use is taught to occur with the knowledge that a patient ingesting either of two tablettes formed from, e.g., a 40 mg tablet, could be ingesting a predictable 20 mg dose that would represent a dose as acceptable to regulators such as the U.S. Food and Drug Administration “FDA”) as in an individually made 20 mg tablet.

Recently, Solomon and Kaplan have disclosed novel dosage forms that comprise a layered tablet structure containing a preferably pharmacologically inactive segment that serves as a preferred breaking region if tablet subdivision is desired. See, e.g., WO 2005/112898 and WO 2005/112900.

It is now recognized that the subject methods of treatment of medical conditions may be employed, preferably by utilizing the novel dosage forms of the above inventions, because accurate and predictable divided doses are taught to be created. These compositions and methods can be incorporated in medical, nursing, or institutional, treatment plans, and the like, as well as in PI's for drug products.

### DESCRIPTION OF THE INVENTION

It is a primary object of the invention to provide a method of treating patients to a numerical goal such as a desired blood pressure, cholesterol level, or thyroid-stimulating hormone level, or to reach a desired clinical or therapeutic endpoint, such as reduction in anxiety, depression, or asthma, or seizures. The method of the invention can be utilized in the treatment of chronic conditions such as hypertension and hyperlipidemia. The invention involves the use of finished dosage forms, e.g., tablets, by breaking or otherwise dividing them to produce a predictably accurate smaller or lower dose (or sub-dose, also referred to herein as a fractional dose). This advantageously allows tablet breaking to be recommended and even preferred in treatment plans, authoritative guidelines, manufacturer's product information, and the like.

The above-mentioned published International patent applications WO 2005/112898 and WO 2005/112900 disclose preferred dosage forms that allow this improvement in the medical and pharmaceutical arts. However, the method of the subject invention is not limited to the use such dosage forms and can be carried out using any finished dosage form capable of being accurately broken or divided to provide a predictable lower dose. It would also be understood that the fractional dose provided by breaking of the finished dosage form (forming, for example, a tablette, as defined in the above-referenced international applications) may be further subdivided or broken to provide yet a lower dose in the resulting portions thereof. These further subdivisions, which are also breakable into predictably accurate doses, and the methods of use associated therewith, are within the scope of the subject invention.

Preferably, instructions and use of the subject compositions and methods involve upward dose titration, such as are commonly used for medical treatment of disorders involving cholesterol, diabetes mellitus, hypertension, thyroid disorders, and epilepsy. Also within the scope of the invention are downward dose titration regimens such as are used with treatment of acute allergic or asthmatic reaction using prednisone or in anti-epileptic treatments with a second medication while decreasing the dose of a first medication.

A broader embodiment of the invention involves treatment plans and other usage that utilize alternating doses or doses that are in between the doses of available whole dosage forms. For example, for a product such as Lamictal that is only available in the U.S. in 25 and 100 mg strength tablets, the compositions and methods of the subject invention can provide for a 37.5 mg dose by accurately and predictably breaking a 25 mg tablet into two tablettes, each containing 12.5 mg, then administering one 25 mg tablet and one 12.5 mg tablette (1½ of the 25 mg tablets). Advantageously, the subject invention provides that administering three 12.5 mg tablettes will be the equivalent of the above-described dosing using one 25 mg tablet plus one 12.5 mg tablette.

A related embodiment of the invention involves clinical situations in which, for example, a nurse in a hospital has received and intends to administer to a patient a unit dose of a 20 mg lisinopril tablet, but before administration of this tablet, a treating physician writes an order to lower the dose to 10 mg. With the method of the invention, the nurse may comply with the order by breaking a 20 mg tablet that provides an accurate, predictable 10 mg dose, such as a 20 mg tablet produced, for example, according to the teachings of Solomon and Kaplan. With the current art, whether or not a lisinopril tablet were scored, it would not be feasible to provide a half tablet with the reasonable assurance that said half tablet would be equivalent to an individually manufactured 10 mg lisinopril tablet, especially given the uncertainty of who is breaking the 20 mg tablet and under what conditions.

In the treatment of hypercholesterolemia, current methods employed in the U.S. include beginning with the maximum dose that is hoped by the prescriber to be tolerated, taking into account such factors as a patient's age, weight, hepatic function, cholesterol level, and/or current and desired cholesterol levels. Because potential side effects such as myalgias and rhabdomyolysis are dose dependent, physicians tend to start with a lower “safe” dose, even if that dose is predicted to be inadequate to bring a patient to a desired goal. Because all statins marketed in the U.S. are currently unscored, are likely to be difficult to break and therefore provide unpredictable doses if broken or otherwise divided, physicians often initially prescribe a lower dose than they expect to ultimately be needed. Thus atorvastatin may be prescribed at 10 mg daily, then, as shown to be tolerated by the patient, new prescriptions may be subsequently given for 20 mg and then 40 mg tablets. This dosing routine may require multiple visits to a physician or pharmacy, additional costs, and other disadvantageous aspects.

In accordance with the subject invention, dosing may begin with one-quarter of a 40 mg tablet, which predictably provides an accurate 10 mg dose. Then, when tolerability of this 10 mg dose is demonstrated, such as after 4 or 8 days, dosing may increase to one-half of the 40 mg tablet daily (providing a predictable 20 mg dose), and potentially then to one 40 mg tablet daily. Advantageously, using the compositions and methods of the subject invention, it is expected that patient compliance with the ultimate goal of dosing 40 mg daily will be increased, as compared to purchasing three separate tablet strengths, for reasons that include the starting dose appearing cautiously low at only ¼ of a tablet daily. Moreover, the compositions and methods of the subject invention are advantageous in that fewer visits to the physician or receipt of prescriptions from the physician for any one patient are required. A preferred means of utilizing the invention as in this example of a quadrisected statin tablet is to utilize the inventions described in International Application, WO 2005/112900.

It is also expected that the method of the invention will increase the percentage of physicians who get patients to goal. It has been well documented that many physicians under-treat generally asymptomatic conditions such as elevated hyperlipidemia, so that having a high dose that can be initially used as a low dose is likely to increase the number of physicians who utilize that dose. Not only do compositions and methods of the subject invention allow titration upwards to occur in a fully acceptable manner, but if a patient receives a dosage that is double the starting dose, an accurately breakable dosage form allows the patient to go to, for example, a half dose (i.e., said starting dose) if a side effect appears at the higher dose that was not present at the starting dose.

In the treatment of hypertension, whether essential or secondary, it is recognized that many patients are sensitive to medication commonly prescribed to treat this condition. It is also recognized that many patients require higher doses than the starting doses, even if their degree of hypertension is (at least initially) considered by the physician to be mild (Stage 1). Thus, it is common to begin with a starting dose of a medication below that expected to be needed as a final or maintenance dose. In the invention, a predictable starting dose that is a fraction of a larger dose is utilized for an adequate period of time to demonstrate tolerability and safety, and to evaluate adequacy of dosing, then dosing is increased by providing either the whole tablet or a larger fraction of the dose thereof. Beginning treatment of hypertension with lisinopril 2.5 mg, as one-quarter of a quadrisected 10 mg dose is an example of the invention, with increasing dosing to 5 mg (one-half of a scored 10 mg dose), and then to 7.5 or 10 mg, using accurately divided portions (e.g., tablettes) and/or whole tablets as needed.

In another example, glyburide is currently typically provided as scored 1.25, 2.5 and 5 mg tablets. Dosing is up to 20 mg daily in either once or twice daily doses. In the PI for drug products containing glyburide as the active ingredient, dose titration is mentioned but no instruction to split the scored tablets is expressly recited or otherwise provided. In accordance with the subject invention, the use of a scored 2.5 mg tablet which is predictably and accurately divisible into two 1.25 mg tablettes can be employed to achieve a daily dose of 3.75 mg without having to purchase a 1.25 mg tablet in addition to the 2.5 mg tablet. In accordance with the subject invention, a predictably and accurately breakable 5 or 10 mg quadrisected tablet may also be utilized. Dosing in this example begins using ¼ tablet daily and may increase to the use of half tablets or a combination of a quarter-, half- or whole tablet as tolerated and as glycemic response necessitates. Additionally, the initial tablet utilized could be a 4, 6 or 8 mg tablet. There is no requirement in the subject invention that introduction of a new version and/or a new method of administration requires dosing to utilize the same doses as were previously available.

In another example, Synthroid® (L-thyroxine) is currently marketed in the U.S. as a scored tablet. Again, despite the presence of a score, no PI instruction exists to utilize the drug as a half-tablet. Dosing per the invention may, for example, utilize a 100 mcg quadrisected tablet, with instructions to the patient to begin using ¼ tablet (predictably providing a tablette containing an accurate 25 mcg dose) daily for 8 days (a total of two 100 mcg tablets), then ½ tablet daily (predictably providing a tablette containing an accurate 50 mcg dose) for 24 days (i.e., administering a total of six 100 mcg tablets), then to have an office visit and blood test to determine the appropriateness of a further dose increase to 75 or 100 mcg. Thus, a single prescription and two visits to the physician are required by this dosing schedule using a dosage form which is breakable into predictably accurate doses. By contrast, previous dosing schedules would require at least three prescriptions (one each for 25, 50, and 75 or 100 mcg tablets, and often a physician visit for each new prescription. Therefore, prescription costs and medical expenses such as physician visitation costs can be substantially decreased by employing compositions and methods in accordance with the subject invention.

Another example involves dosing schedules using an alpha-adrenergic blocking agent, such as doxazosin, terazosin, or prazosin. The mandated starting dose for each is 1 mg, preferably taken before bed. The above alpha-adrenergic blocking agent drugs are indicated for hypertension and also for benign prostatic hypertrophy/hyperplasia (“BPH”). In accordance with the method of the subject invention, an accurately breakable bisected 2 mg tablet (accurately yielding two 1 mg tablettes), trisected 3 mg tablet (accurately yielding three 1 mg tablettes, of quadrisected 4 mg tablet (accurately yielding four 1 mg tablettes) may be used to provide the 1 mg starting dose, and then upward dose adjustment would be made using the tablet of the invention, and tablettes created therefrom, as medically appropriate.

Management of anxiety and pain also can benefit from the procedures of the invention. A physician can devise, and a PI can authorize or instruct, a treatment regimen that for example could comprise 0.5 mg (one tablet) of alprazolam nightly and 0.25 mg (=±½ tablet) as needed during the day.

In yet a different embodiment of the example, a drug such as prednisone is commonly utilized to treat a condition such as an acute allergic or asthmatic reaction. In one of many potential examples of the usefulness of the invention, a 40 mg quadrisected prednisone tablet may come to be created which is predictably breakable into tablettes containing accurate 20 mg (halved) or 10 mg (quartered) doses, and this 40 mg tablet can be prescribed for asthma. The subject method comprises a treatment whereby the patient is initially administered a whole 40 mg tablet, then per physician's instruction, the dose may be reduced to 30 mg (¾ of a predictably and accurately breakable 40 mg tablet), or 20 mg (one-half of a predictably and accurately breakable 40 mg tablet), then administering 10 mg (¼ of a predictably and accurately breakable 40 mg tablet). The treatment method can then include administration of a dose as directed by the physician. After the patient reaches the 10 mg dose (¼ tablet), the invention may be utilized again by prescribing a sub-10 mg bisected, trisected, or quadrisected dose and having the patient taper off the medication by utilizing progressively smaller fractions of the whole dose.

In a different embodiment of this de-escalation dosing regimen in accordance with the invention, an acute allergic reaction involving laryngeal edema may be treated using two 40 mg tablets, each quadrisected, then lowering the dose by 10-20 mg per day as directed utilizing whole tablets that predictably and accurately breakable into tablettes containing a fraction of the dose in the whole tablet, or fractions thereof.

In the above examples, quadrisection and dosing using a defined portion of a whole tablet is provided as a preferred embodiment of the invention. No limitation is intended. For example, a tablet may be sectioned to provide more than four sections. These and other embodiments of the invention that are contemplated by or suggested to persons of ordinary skill in the art using this disclosure and/or which is known within the art is within the scope of the invention. Alternatively, fewer than four sections of a tablet may be formed. For example, when bisection (providing two halves) is adequate to allow dose titration and adjustment, then such is also within the scope of the invention.

Further, it is preferred that accurate division of a tablet with regard to the fractional doses be obtained, such as, but not limited to the dosage forms described in published International patent applications WO 2005/112898 and WO 2005/112900 and other means of creating pharmaceutical tablets optimized for accurate breaking using known, homogeneous tablets, including elongated tablets.

Among the benefits of the subject invention are improvements in patient compliance. Patients typically prefer taking a fraction of a dose to start treatment and then increasing the dose by taking a larger portion of the tablet and/or the whole tablet. Patient compliance can be enhanced especially for asymptomatic conditions such as hypertension and hypercholesterolemia. Patient compliance is also expected to be enhanced by a decrease in the number of prescriptions required.

From a physician's standpoint, or that of other prescribers, the level of complexity of prescribing may advantageously be decreased by the methods of the invention. By use of compositions or methods of the subject invention, a prescriber can recommend safe usage of a partial dose, such as in a titration dosing scheme without concern of inaccurate doses resulting from breaking of a tablet where the inaccurate dose may not protect the patient, or may cause unwanted side effects, such as can happen in a dose-dependent way in epilepsy, diabetes, hypertension, and the like.

More generally, the prescriber can also authorize a patient to achieve a predictable dose outside a titration dosing scheme. For example, a patient whose use of the drug lamotrigine (currently available only in 100 and 25 mg doses), as described earlier, could involve administration of 100 mg in the morning and 50 mg (one-half tablet) in the evening or before bed. Use of the currently available dosage forms would, per the label (Patient Instruction), may require two separate prescriptions, prescribed as one 100 mg tablet and a separately prescribed 25 mg tablet (two tablets for the 50 mg dose) each day. Per the invention, the patient could safely utilize one whole and one-half 100 mg tablet daily. Such use can therefore be advantageous for physicians as well as patients.

The subject invention applies to methods and compositions useful for treatment of animals, preferably mammals, and more preferably to humans.

It would be readily understood that the compositions useful for application to the methods in accordance with the subject invention may be scored or unscored. It would be further understood that the term “bisected” dosage form or tablet refers to the dosage form bearing one or more marks or scores indicating divisibility of that dosage form into two portions or fractional doses, preferably two equal portions or halves. The bisecting mark or score may be a single line, for example a score line collinear with the diameter of a conventional round tablet, or may be transverse to the longest dimension of a capsule-shaped tablet. Similarly, references herein to a “trisected” dosage form or tablet identifies a dosage form bearing one or more marks or scores that indicate division of the dosage form into three portions, preferably three equal portions or thirds. “Quadrisected,” accordingly, refers to at least one mark or score in or on a dosage form that indicates division of the dosage form into four portions, preferably four equal portions or quarters. Further numbers of divisions of a dosage form into five or more portions or fractional doses may also be employed in the subject invention, and may only be limited by the result of requiring a predictably accurate lower dose upon such division of the dosage form.

Moreover, the subject invention further includes an article of manufacture, or kit, that comprises a finished dosage form which is breakable or otherwise divisible into a predictably accurate lower dose, and an instruction, preferably a written or electronic instruction for breaking the dosage form and administering a fractional dose of said finished dosage form, or a divided dosage form. Preferably, the article of manufacture includes a packaged tablet or tablets and a separate instruction for use in accordance with the subject method.

It is recognized that related inventions may be within the spirit of the disclosures herein. Also no omission in the current application is intended to limit the inventors to the current claims or disclosures. While certain preferred and alternative embodiments of the invention have been set forth for purposes of disclosing the invention, modifications to the disclosed embodiments may occur to those who are skilled in the art.

1-36. (canceled)

37. A method of treating a medical condition in which a prescribed dose is provided by breaking a dosage form containing a dose of a drug or pharmaceutical agent to provide at least two portions of said dosage form that each comprise a predictable and accurate lower dose of said drug or pharmaceutical agent, said medical condition being exhibited by an animal, wherein said dosage form is segmented and comprises at least two active segments comprising an active pharmaceutical ingredient, and at least one segment which is substantially pharmaceutically inactive, said substantially pharmaceutically inactive segment interposed between the at least two active pharmaceutical ingredient-containing segments.

38. A method of treating a medical condition by initiating treatment utilizing a predictable fractional dose, said fractional dose obtained by breaking the finished dosage form to create said fractional dose, wherein said dosage form is segmented and comprises at least two active segments comprising an active pharmaceutical ingredient, and at least one segment which is substantially pharmaceutically inactive, said substantially pharmaceutically inactive segment interposed between the at least two active pharmaceutical ingredient-containing segments.

39. The method of claim 38 wherein said fractional dose is selected from the group consisting of a half, a third, a quarter, and a fifth of a dose contained in a finished dosage form,

40. The method of claim 38 in which said finished dosage form comprises a tablet that can be broken accurately to provide a predictable fractional dose with regard to the drug or drugs or other pharmaceutical agents contained therein.

41. The method of claim 37 wherein said dosage form is unscored.

42. The method of claim 37 wherein said dosage form comprises a score or other type of separation mark for guiding a user to break said dosage form in an area marked by said score or separation mark.

43. The method of claim 37 in which said dosage form comprises a plurality of unitary segments.

44. The method of claim 37 in which the dosage form comprises a plurality of adhesively joined preformed subunits.

45. The method of claim 44 in which the dosage form comprises a pharmacologically inactive subunit and a pharmaceutically active subunit.

46. The method of claim 37 in which said medical condition comprises at least one of hypertension, hyperlipidemia, diabetes mellitus, hypothyroidism, benign prostatic hyperplasia, asthma, or allergic condition.

47. The method of claim 37 comprising dose de-escalation to treat a medical condition.

48. The method of claim 37 comprising dose escalation to treat a medical condition.

49. The method of claim 37 wherein said dosage form comprises a steroid.

50. A method for reaching a desired therapeutic or clinical end-point, said method comprising providing a segmented dosage form comprising at least two active segments which comprise an active pharmaceutical ingredient, and at least one segment which is substantially pharmaceutically inactive, said substantially pharmaceutically inactive segment being interposed between the at least two active pharmaceutical ingredient-containing segments, breaking the dosage form into a second, smaller dosage form that contains a smaller dose, and administering said fractional dose as an initial dose.

51. The method of claim 50 in which said breaking of said dosage form produces a predictable smaller dose.

52. A method for improving patient compliance in dose administration of a medication, said method comprising providing a segmented dosage form comprising at least two active segments which comprise an active pharmaceutical ingredient, and at least one segment which is substantially pharmaceutically inactive, said substantially pharmaceutically inactive segment being interposed between the at least two active pharmaceutical ingredient-containing segments, breaking the dosage form into a second, smaller dosage form that contains a smaller dose, and administering said fractional dose as an initial dose.

53. The method of claim 52 wherein said dosage form comprises a tablet.

54. The method of claim 53 wherein said tablet comprises a layered tablet.

55. An article of manufacture or kit comprising medication in a first dosage form comprising at least two active segments which comprise an active pharmaceutical ingredient, and at least one segment which is substantially pharmaceutically inactive, said substantially pharmaceutically inactive segment being interposed between the at least two active pharmaceutical ingredient-containing segments, wherein said dosage form is breakable into a second, smaller dosage form that contains a predictable, smaller dose, said article of manufacture or kit further comprising a separate instruction to create a smaller dosage by breaking said first dosage form.
